(19) United States
(12) Patent Application Publication
(10) Pub. No.: US 2002/0091097 A1
(43) Pub. Date: Jul. 11, 2002

(54) Nucleic acids for the prevention and treatment of sexually transmitted diseases

(76) Inventors: Robert L. Bratzler, Concord, MA (US); Deanna M. Petersen, Newton, MA (US)

Correspondence Address: Maria A. Trevisan, c/o Wolf, Greenfield & Sacks, P.C., Federal Reserve Plaza, 600 Atlantic Avenue, Boston, MA, 02210-2211, US

(21) Appl. No.: 09/949,194

(22) Filed: Sep. 7, 2001

Related U.S. Application Data
(60) Provisional application No. 60/230,637, filed on Sep. 7, 2000.

(51) Int. Cl. A61K 48/00

(52) U.S. Cl. 514/44

(57) ABSTRACT

The invention relates to methods and products for preventing and/or treating sexually transmitted diseases. A nucleic acid and optionally an anti-STD agent, a birth control agent and/or a birth control device are administered, optionally in the context of a sustained release device to a subject to prevent or treat STD.

### PRIORITY OF THE INVENTION

This application claims priority under Title 35 §119(e), of United States Provisional Application No. 60/230,637, filed Sep. 7, 2000, entitled NUCLEIC ACIDS FOR THE PREVENTION AND TREATMENT OF SEXUALLY TRANSMITTED DISEASES, the entire contents of which are incorporated herein by reference.

### FIELD OF THE INVENTION

The invention relates to methods, products, and kits for treating and/or preventing sexually transmitted diseases.

BACKGROUND OF THE INVENTION

Millions of individuals worldwide suffer from sexually transmitted diseases (STDs), which are generally bacterial, viral or parasite infections transferred between persons through sexual contact. In the past, STDs such as gonorrhea and syphilis were readily treatable with antibiotics such as penicillin. However, more recently, some forms of STDs, such as Herpes and Hepatitis B, have been recognized which cannot be cured effectively. In addition, many types of STD-causing pathogens have developed resistance to commonly used antibiotics (e.g., penicillin resistant gonorrhea).

### SUMMARY OF THE INVENTION

The invention is based, in part, on the discovery of a new class of compounds for the treatment and prevention of sexually transmitted disease (STD). The invention, in one aspect, is a method for preventing or treating an STD by administering to a subject in need thereof a nucleic acid in an amount effective to prevent or treat an STD.

The invention provides, in another aspect, a method for preventing or treating an STD which involves administering to a subject in need thereof a nucleic acid in an amount effective to induce an immune response at a local site in the subject.

In one aspect, a method is provided for preventing or treating a sexually transmitted disease comprising administering to a subject in need thereof a poly-G nucleic acid in an amount effective to induce an immune response at a local site in the subject, wherein the subject is at risk of exposure at the local site to an agent that causes the sexually transmitted disease selected from the group consisting of Neisseria gonorrhoeae, Chlamydia trachomatis, Treponema pallidum, Haemophilus ducreyi, Condyloma acuminata, Calymmatobacterium granulomatis, Ureaplasma urealyticum, Human T lymphotropic virus type I (HTLV-I), Human papilloma virus (multiple types), Hepatitis B virus, Molluscum contagiosum virus, Trichomonas vaginalis, Phthirus pubis, Candida albicans, Mycoplasma hominis, Gardnerella vaginalis and Group B streptococcus, Human T lymphotrophic virus type II (HTLV-II), Hepatitis C and D viruses, Sarcoptes scabiei, Shigella spp., Campylobacter spp., Hepatitis A virus, Giardia lamblia and Entamoeba histolytica.

In another aspect, a method is provided for preventing or treating a sexually transmitted disease comprising administering to a subject in need thereof and not actively exposed to an antigen a poly-G nucleic acid in an amount effective to induce an immune response at a local site in the subject, wherein the subject is at risk of exposure at the local site to an agent that causes the sexually transmitted disease selected from the group consisting of Herpes simplex virus types 1 and 2 (HSV-1 and HSV-2), Hepatitis C and D viruses, and Epstein-Barr virus (EBV).

In yet another aspect, a method is provided for preventing or treating a sexually transmitted disease comprising administering to a subject in need thereof a non-motif phosphorothioate nucleic acid in an amount effective to induce an immune response at a non-skin local site in the subject, wherein the subject is at risk of exposure at the local site to an agent that causes the sexually transmitted disease selected from the group consisting of Neisseria gonorrhoeae, Chlamydia trachomatis, Treponema pallidum, Haemophilus ducreyi, Condyloma acuminata, Calymmatobacterium granulomatis, Shigella spp., Ureaplasma urealyticum, Mycoplasma hominis, Gardnerella vaginalis, Campylobacter spp., Group B streptococcus, Human immunodeficiency viruses (HIV-1 and HIV-2), Human T lymphotropic virus type I (HTLV-1), Human T lymphotrophic virus type II (HTLV-II), Herpes simplex virus type I (HSV-1) Herpes simplex virus type 2 (HSV-2), Human papilloma virus (multiple types), Hepatitis A virus, Hepatitis B virus, Hepatitis C and D viruses, Epstein-Barr virus (EBV), Cytomegalovirus and Molluscum contagiosum virus, Trichomonas vaginalis, Sarcoptes scabiei, Giardia lamblia, Phthirus pubis, Entamoeba histolytica and Candida albicans.

In a further aspect, a method is provided for preventing or treating a sexually transmitted disease comprising administering to a subject in need thereof a non-motif phosphorothioate nucleic acid in an amount effective to induce an immune response at a local site in the subject, wherein the subject is at risk of exposure at the local site to an agent that causes the sexually transmitted disease selected from the group consisting of Neisseria gonorrhoeae, Chlamydia trachomatis, Treponema pallidum, Haemophilus ducreyi, Condyloma acuminata, Calymmatobacterium granulomatis, Shigella spp., Ureaplasma urealyticum, Mycoplasma hominis, Gardnerella vaginalis, Campylobacter spp., Group B streptococcus, Human immunodeficiency viruses (HIV-1 and HIV-2), Human T lymphotropic virus type I (HTLV-I), Human T lymphotrophic virus type II (HTLV-II), Hepatitis A virus, Hepatitis B virus, Hepatitis C and D viruses, Epstein-Barr virus (EBV), Cytomegalovirus and Molluscum contagiosum virus, Trichomonas vaginalis, Sarcoptes scabiei, Giardia lamblia, Phthirus pubis, Entamoeba histolytica and Candida albicans.

In another aspect, a method is provided for preventing or treating a sexually transmitted disease, comprising administering to a subject in need thereof a nucleic acid in an amount effective to induce an immune response at a local site in the subject, wherein the subject is at risk of exposure at the local site to an agent that causes the sexually transmitted disease selected from the group consisting of Haemophilus ducreyi, Calymmatobacterium granulomatis, Ureaplasma urealyticum, Gardnerella vaginalis, Shigella spp., Molluscum contagiosum virus, Epstein-Barr virus, Trichomonas vaginalis, Phthirus pubis, Giardia lamblia, Entamoeba histolytica, and Sarcoptes scabiei.

Preferably, the subject is at risk of exposure to an agent that causes the STD at the local site, be it skin or non-skin local site. The nucleic acid may be administered to the subject prior to engaging in a high risk activity, during engagement in a high risk activity or following engagement in a high risk activity. Administration prior to engaging in a high risk activity includes but is not limited to at least one month, at least one week, at least 48 hours, at least 24 hours, at least 12 hours, at least 6 hours, at least 4 hours, and at least 2 hours prior to engaging in the high risk activity such as, for example, sex. Administration following engagement in the high risk activity includes but is not limited to within 2 hours, within 4 hours, within 6 hours, within 12 hours, within 24 hours, within 48 hours, or within 3, 4, 5, 6, 7, 14, 28 days or longer after engaging in the high risk activity.

The high risk activity may be selected from the group consisting of sexual intercourse (e.g., oral, vaginal or anal), blood transfusion, intravenous needle use, childbirth, and certain medical procedures (particularly those involving contact with bodily fluids), but are not so limited. In embodiments in which the high risk activity is a blood transfusion, the nucleic acid may be coated on an inside surface of a transfusion (i.e., intravenous) bag, an intravenous tube, or an intravenous needle, or may be provided within the intravenous bag to be dissolved in the intravenous solution. In embodiments in which the high risk activity is sexual intercourse, the nucleic acid may be coated on a birth control device such as a condom (male and female), an intrauterine device, an intra-vaginal device, a cervical cap and a contraceptive sponge.

In other embodiments, the nucleic acid is administered systemically.

In some preferred embodiments, the nucleic acid is delivered in the absence of antigen exposure of the subject. In some embodiments relating to mucosal delivery of the nucleic acid, the subject is not exposed to an agent that causes a sexually transmitted disease.

The invention intends to prevent or treat STDs including, but not limited to, HIV/AIDS, chancroid, chlamydia, gonorrhea, hepatitis, herpes simplex virus I and II, syphilis, trichomonas, venereal warts, and candida. In another aspect, the invention also aims to prevent and treat some STD related conditions, such as pelvic inflammatory disease, by administering the nucleic acids of the invention. In yet other embodiments, pubic lice and scabies are prevented or treated using the nucleic acids described herein.

A nucleic acid is an element of each aspect of the invention. The nucleic acids useful according to the invention may be synthetic or natural nucleic acids. In some preferred embodiments, the nucleic acids are isolated or substantially purified.

In one embodiment, the nucleic acid is an immunostimulatory nucleic acid. The immunostimulatory nucleic acid is any nucleic acid which is capable of modulating an immune response. In some embodiments, the immunostimulatory nucleic acid is a CpG nucleic acid having an unmethylated CpG motif (particularly an unmethylated C in a CpG dinucleotide), a T-rich nucleic acid (including a poly T nucleic acid), a poly G nucleic acid, or a methylated CpG nucleic acid having a methylated CpG motif. In some embodiments, the immunostimulatory nucleic acid is not an antisense nucleic acid specific to the genes of an STD-causing pathogen. In still other embodiments, the nucleic acid is not a vector that encodes a peptide or polypeptide such as an antigen from an STD-causing pathogen. In other embodiments, the immunostimulatory nucleic acid is an antisense nucleic acid or a vector expressing a gene encoding an antigen from an STD-causing pathogen, provided it is capable of stimulating an immune response independent of its antisense or antigen encoding capability. As an example, the immunostimulatory motifs described herein may be incorporated into a nucleic acid which is otherwise an antisense nucleic acid or a nucleic acid which encodes an antigen.

The nucleic acid, in some embodiments, has a nucleotide backbone which includes at least one backbone modification, such as a phosphorothioate modification or other phosphate modification. In some embodiments, nucleic acids having a phosphorothioate backbone modification are not intended for use in the prevention and treatment of infection of skin cells by HSV-1, HSV-2 and HPV (e.g., condyloma acuminata lesions of the skin). “Non-motif” phosphorothioate nucleic acids are nucleic acids having at least one phosphorothioate backbone modification and lacking an immunostimulatory motif selected from the group of unmethylated CpG motif, a methylated CpG motif, T-rich motif and poly G motif. These nucleic acids can be used to prevent and/or treat a range of STD described herein.

In some embodiments, the modified backbone is a peptide modified oligonucleotide backbone. The nucleotide backbone may be chimeric, or the nucleotide backbone may be entirely modified.

The immunostimulatory nucleic acid can have any length greater than 6 nucleotides, but, in some embodiments, is between 8 and 100 nucleotide residues in length. In another embodiment, the nucleic acid may be between 8 and 40 nucleotides in length. In other embodiments, the nucleic acid comprises at least 20 nucleotides, at least 24 nucleotides, at least 27 nucleotides, or at least 30 nucleotides. The nucleic acid may be single stranded or double stranded. In some embodiments, the nucleic acid is isolated and in other embodiments, the nucleic acid may be a synthetic nucleic acid.

The CpG nucleic acid, in one embodiment, contains at least one unmethylated CpG dinucleotide having a sequence including at least the following formula: 5′ X₁ X₂CGX₃ X₄ 3′ wherein C is unmethylated, wherein X₁, X₂, X₃, and X4 are nucleotides. In another embodiment, the methylated CpG nucleic acid comprises: 5′ X₁ X₂CGX₃ X₄ 3′ wherein C is methylated, wherein X₁, X₂, X₃, and X₄ are nucleotides. In one embodiment, the 5′ X₁ X₂CGX₃ X₄ 3′ sequence of the CpG nucleic acid or the methylated CpG nucleic acid is a non-palindromic sequence, and in other embodiments, it is a palindromic sequence.

In some embodiments, X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA. In other embodiments, X₁X₂ are GpA or GpT and X₃X₄ are TpT. In yet other embodiments, X₁ or X₂ or both are purines and X₃ or X₄ or both are pyrimidines or X₁X₂ are GpA and X₃ or X₄ or both are pyrimidines. In one embodiment, X₂ is a T and X₃ is a pyrimidine.

In some embodiments, the T-rich immunostimulatory nucleic acid is a poly T nucleic acid comprising 5′ TTTT 3′. In yet other embodiments, the poly T nucleic acid comprises 5′ X₁ X₂TTTTX₃ X₄ 3′ wherein X₁X₂, X₃ and X4 are nucleotides. In some embodiments, X₁X₂ is TT and/or X₃X₄ is TT. In other embodiments, X₁X₂ is selected from the group consisting of TA, TG, TC, AT, AA, AG, AC, CT, CC, CA, CG, GT, GG, GA, and GC; and/or X₃X₄ is selected from the group consisting of TA, TG, TC, AT, AA, AG, AC, CT, CC, CA, CG, GT, GG, GA, and GC.

The T-rich immunostimulatory nucleic acid may have only a single poly T motif or it may have a plurality of poly T nucleic acid motifs. In some embodiments, the T-rich immunostimulatory nucleic acid comprises at least 2, at least 3, at least 4, at least 5, at least 6, at least 7, or at least 8 poly T motifs. In other embodiments, it comprises at least 2, at least 3, at least 4, at least 5, at least 6, at least 7, or at least 8 CpG motifs. In some embodiments, the plurality of CpG motifs and poly T motifs are interspersed.

In yet other embodiments, at least one of the plurality of poly T motifs comprises at least 3, at least 4, at least 5, at least 6, at least 7, at least 8, or at least 9 contiguous T nucleotide residues. In other embodiments, the plurality of poly T motifs is at least 3 motifs and wherein at least 3 motifs each comprises at least 3 contiguous T nucleotide residues or the plurality of poly T motifs is at least 4 motifs and wherein the at least 4 motifs each comprises at least 3 contiguous T nucleotide residues.

The T-rich immunostimulatory nucleic acid may include one or more CpG motifs. The motifs may be methylated or unmethylated. In other embodiments, the T-rich immunostimulatory nucleic acid is free of one or more CpG dinucleotides.

In other embodiments, the T-rich immunostimulatory nucleic acid has poly A, poly G, and/or poly C motifs. In other embodiments, the T-rich immunostimulatory nucleic acid is free of two poly C sequences of at least 3 contiguous C nucleotide residues. Preferably the T-rich immunostimulatory nucleic acid is free of two poly A sequences of at least 3 contiguous A nucleotide residues. In other embodiments, the T-rich immunostimulatory nucleic acid comprises a nucleotide composition of greater than 25% C or greater than 25% A. In yet other embodiments, the T-rich immunostimulatory nucleic acid is free of poly C sequences, poly G sequences or poly-A sequences.

In some cases the T-rich immunostimulatory nucleic acid may be free of poly T motifs, but rather, may comprise a nucleotide composition of greater than 25% T. In other embodiments, the T-rich immunostimulatory nucleic acid may have poly T motifs and may also comprise a nucleotide composition of greater than 25% T. In some embodiments, the T-rich immunostimulatory nucleic acid comprises a nucleotide composition of greater than 25% T, greater than 30% T, greater than 40% T, greater than 50% T, greater than 60% T, greater than 80% T, or greater than 90% T nucleotide residues. In other embodiments, the T-rich nucleic acids are at least 20 nucleotides in length or at least 24 nucleotides in length.

Examples of T rich nucleic acids that are free of CpG nucleic acids and of T rich nucleic acids that include CpG nucleic acids are described in U.S. Non-Provisional Patent Application Serial No. 09/669,187, filed Sep. 25, 2000.

In some embodiments, the poly G nucleic acid comprises: 5′ X₁X₂GGGX₃X₄ 3′ wherein X₁, X₂ X₃, and X₄ are nucleotides. In other embodiments, at least one of X₃ and X₄ are a G or both of X₃ and X₄ are a G. In still other embodiments, the poly G nucleic acid comprises the following formula: 5′ GGGNGGG 3′ wherein N represents between 0 and 20 nucleotides. In yet other embodiments, the poly G nucleic acid comprises the following formula: 5′ GGGNGGGNGGG 3′ wherein N represents between 0 and 20 nucleotides.

The poly G immunostimulatory nucleic acid may include one or more CpG motifs or T-rich motifs. The CpG motifs may be methylated or unmethylated. The poly G nucleic acid may include at least one unmethylated CpG dinucleotide. In other embodiments, the poly G nucleic acid is free of one or more CpG dinucleotides or T-rich motifs.

In some embodiments the poly G nucleic acid is free of unmethylated CG dinucleotides, such as, for example, the nucleic acids described in U.S. Non-Provisional Patent Application Serial No. 09/669,187, filed Sep. 25, 2000. In other embodiments the poly G nucleic acid includes at least one unmethylated CG dinucleotide, such as, for example, the nucleic acids described in U.S. Non-Provisional Patent Application Serial No. 09/669,187, filed Sep. 25, 2000.

In some embodiments, the nucleic acid is capable of stimulating a Th1 immune response. In other embodiments, the nucleic acid is not one which is capable of inducing a Th2 immune response (i.e., the nucleic acid is a Th2 immunostimulatory nucleic acid). In still other embodiments, the nucleic acid may be a Th2 immunostimulatory nucleic acid provided it is administered by a non-mucosal route. In some embodiments, Th2 immunostimulatory nucleic acids are used in the prevention and treatment of bacterial sexually transmitted diseases. According to yet a further embodiment, the nucleic acids of the invention are administered in a dose, an administration route and a schedule which induces a Th1 response.

The nucleic acid may be administered alone or in conjunction with a pharmaceutically-acceptable carrier and optionally other therapeutic agents. Other therapeutic agents are preferably non-nucleic acid therapeutic agents and include, but are not limited to, anti-STD agents, non-drug anti-STD agents, birth control agents and mucosal adjuvants.

In one aspect, the invention provides a method for preventing or treating an STD by administering, to a subject in need thereof, a nucleic acid and an anti-STD agent in an effective amount to prevent or treat an STD.

Anti-STD agents include anti-bacterial agents, anti-viral agents, anti-fungal agents and anti-parasitic agents, but are not so limited. The anti-bacterial agent may be an antibiotic, such as a broad spectrum antibiotic, a narrow spectrum antibiotic, or a limited spectrum antibiotic. In some embodiments, the anti-bacterial agent is a cell wall synthesis inhibitor, cell membrane inhibitor, protein synthesis inhibitor, nucleic acid synthesis or functional or competitive inhibitor. The anti-viral agent may be a compound selected from the group consisting of immunoglobulins, amantadine, interferon, nucleoside analogues, protease inhibitors, trichloroacetic acid, podophyllin, imiquimod, fluorouracil, Acyclovir (Zovirax®), valacyclovir (Valtrex®), famciclovir (Famvir®), but is not so limited.

The invention provides methods and compositions for the prevention or treatment of any of the indicated forms of STDs using the nucleic acids of the invention in combination with a birth control agent or device, and in some instances, an anti-STD agent. The nucleic acid and/or the anti-STD agent may be administered in a birth control device. The birth control device may be selected from the group consisting of a condom (male and female), an intra-uterine device, an intra-vaginal device, a cervical cap, a diaphragm, and a sponge. The nucleic acid and/or the anti-STD agent may be administered with birth control agents such as birth control pills, birth control implants (e.g., Norplant), morning after pills, transdermal patches and spermicides. Birth control agents include both male and female contraceptive agents. In a related aspect, the invention provides a method for preventing or treating STDs other than infections of Candida albicans, HIV or Herpes simplex virus using birth control agents such as transdermal patches.

Another therapeutic agent which may be administered with the nucleic acid is a mucosal adjuvant, particularly if the nucleic acid is administered to a mucosal surface.

In still other embodiments, the nucleic acid, the anti-STD agent and/or optionally the birth control agent may be administered by any route known in the art for delivering medicaments. The medicaments may be administered separately or together, in the same pharmaceutical formulation or separate formulations, by the same route or by different routes. In one embodiment, the nucleic acid is administered on a routine schedule. In another embodiment, the anti-STD agent is administered on a routine schedule. In yet another embodiment, the birth control agent is administered on a routine schedule.

In some embodiments, the local site is selected from the group consisting of the mouth, vagina, anus, penis, eye and blood vessel. In some embodiments, the local site is a non-skin local site. As used herein, a “non-skin local site” is a site on the body that is not skin. Examples of non-skin local sites include internal tissues and mucosal sites.

The nucleic acids and other therapeutic agents may be administered systemically, although in some preferred embodiments, the administration is local. Local administration may include topical application to mucosal surfaces such as those of the mouth, vagina, anus and penis, or to other surfaces such as the lips or skin. In embodiments in which the administration is local, particularly to the mucosal surfaces of the vagina, anus and mouth, the nucleic acid may be one other than a CpG nucleic acid. In particular embodiments, the invention does not intend to prevent or treat human STDs caused by HIV-1, HIV-2, HIV-3, HTLV-I, -II, -III, hepatitis A virus, hepatitis B virus, herpes simplex virus (HSV) 1 and 2, papilloma virus, Neisseria gonorrhoeae, Treponema pallidum, Campylobacter sp., cytomegalovirus (CMV), Chlamydia trachomatis and Candida albicans using local mucosal administration of unmethylated CpG nucleic acids, particularly if the subject is also exposed to antigen from the infectious agent. Embodiments involving the administration of a birth control agent and a nucleic acid of the invention include both local and systemic administration and can induce innate and/or adaptive immunity.

In these and other embodiments, the nucleic acid is administered as a sustained release device. The sustained release device may be situated in an intravenous bag, or it may be a wall of an intravenous bag, or it may be in the wall of the intravenous bag, but it is not so limited. The sustained release device can be polymer or non-polymer based, and can include reverse gel matrices, biodegradable particles including nanoparticles, microparticles, nanospheres, microspheres, nanocapsules and microcapsules, suppositories, pessaries, hydrogels, tampons, rozingers, films, and the like.

In one aspect, the invention provides a non-vacine composition comprising a CpG nucleic acid formulated in a sustained release device in an effective amount, wherein the CpG nucleic acid does not encode a peptide or a polypeptide. In another aspect, the invention provides a composition comprising a nucleic acid selected from the group consisting of a poly-G nucleic acid and a non-motif phosphorothioate nucleic acid, formulated in a sustained release device in an effective amount.

In some embodiments, the nucleic acid and the anti-STD agent can be administered in synergistic combinations. A synergistic combination of a nucleic acid and a birth control agent may also be administered to the subject to prevent or treat an STD particularly if the birth control agent is a spermicidal agent rather a hormone or a barrier method. An example of such a spermicidal agent that is suitable in the synergistic combinations described herein is nonoxynol-9. When synergistic combinations of anti-STD agents and nucleic acids are used, it is preferred, in some embodiments, that the STD is not caused by Neisseria gonorrhoeae, Campylobacter sp., Treponema pallidum, HIV-1, HIV-2, HIV-3, HSV-1, HSV-2, CMV, papilloma virus, Hepatitis A, B and C, HTLV-1, HTLV-II, HTLV-III, Candida albicans or Chlamydia trachomatis. In some embodiments, the invention also does not intend to treat these latter infections if the nucleic acids and other therapeutic agents are staggered in their delivery.

In another aspect, the invention provides a composition, including an effective amount of a nucleic acid for preventing or treating an STD, preferably formulated in a pharmaceutically-acceptable carrier, and a birth control agent. In another embodiment, the birth control agent is selected from the group consisting of a spermicide (such as, for example, nonoxyndol-9) in the form of a foam, gel, lotion, ointment, a vaginal suppository or an anal suppository. In one embodiment, the composition further comprising a birth control device. In important embodiments, the nucleic acid is prepared and administered with a male or female hormonal contraceptive such as a birth control pill, or a birth control implant. In a further embodiment, the composition further comprises an anti-STD agent.

In yet another aspect, the invention provides another composition comprising a nucleic acid formulated in a pharmaceutically-acceptable carrier and in an effective amount for preventing or treating an STD, and a birth control device. This latter composition comprises many if not all of the embodiments of the composition described above.

In a further aspect, the invention provides a composition comprising a nucleic acid, and an intravenous bag, wherein the nucleic acid is situated within the intravenous bag. In one embodiment, the nucleic acid is coated on an inner surface of the intravenous bag. In another embodiment, the nucleic acid is within the intravenous bag. In yet an further embodiment, the nucleic acid is within a wall of the intravenous bag. The invention provides kits containing a nucleic acid, an intravenous bag (as described above) and instructions for use and/or storage.

In yet a further aspect, the invention provides a composition comprising a nucleic acid, and a product which would come into contact with an area likely to develop a yeast infection or to be an area of transmission. Examples of such products include but are not limited to diapers both for children and adults, wipe cloths for cleaning of the genital and anal areas either for children or adults, and ointments such as diaper rash ointments. Other products include tampons and sanitary napkins. In one embodiment, the nucleic acid is contained within or on the surface of the diaper.

According to other aspects the invention provides a kit including a nucleic acid of the invention. The kit may further comprise a sustained release device, a birth control agent, a birth control device, an intravenous bag, a diaper, or some combination thereof. In one embodiment, the kit contains at least one container housing a nucleic acid and the other kit component. The nucleic acid (or some combination of different nucleic acids of the invention) may be housed in the same container or in a different container from the other kit component(s). The kits may further include an anti-STD agent. In of the afore-mentioned kits may further contain instructions for administering the nucleic acid and the other kit components, with or without the anti-STD agent, to a subject having an STD or at risk of developing an STD.

In yet another aspect, the invention provides methods, compositions and kits for preventing or treating a yeast infection in a subject at risk of having or having a yeast infection by administering a nucleic acid of the invention. It is to be understood that any of the foregoing embodiments may be equally applied to these latter aspects of the invention.

Each of the limitations of the invention can encompass various embodiments, of the invention. It is, therefore, anticipated that each of the limitations of the invention involving any one element or combinations of elements can be included in each aspect of the invention.

### DETAILED DESCRIPTION OF THE INVENTION

The invention provides methods and compositions for the prevention and treatment of STDs. As used herein, an STD is an infection which is transmitted primarily, but not exclusively, through sexual intercourse. In addition to being transmitted via sexual contact with an infected subject, some STDs can also be transmitted through contact with bodily fluids of an infected subject. As used herein, “a bodily fluid” includes blood, saliva, semen, vaginal fluids, urine, feces and tears. STDs are most commonly transmitted through blood, saliva, semen and vaginal fluids. As an example, blood and blood product transfusions are common modes of transmission for many sexually transmitted pathogens, including HIV and Hepatitis viruses.

Thus, in one aspect, the invention also intends to embrace the prevention and treatment of classical sexually transmitted diseases even if the particular transmission occurs in a non-sexual manner. As another example, the invention aims to prevent STDs which are transmitted through the use of a contaminated hypodermic needle (e.g., by intravenous drug users, during tattooing or ear piercing). In other embodiments, however, the methods and compositions are intended to prevent and/or treat such infections only if they have been transmitted sexually. STDs may be further defined as infections which are rarely if ever transmitted through agents such as fomites, food, flies or casual contact. Similarly, STDs are not intended to embrace infections from some endogenous pathogens, such as Helicobacter pylori. As used herein, “organism”, “pathogen” and “infectious agent” are used interchangeably to indicate an STD-causing pathogen.

It is common for a subject to have more than one STD or to be at risk of developing more than one STD owing to a particular behavior pattern which is conducive to the transmission of several such pathogens. STDs can be transmitted via heterosexual, homosexual or bisexual activities. STDs can also be transmitted through the sharing of personal hygiene items such as a razor or a toothbrush.

The methods described herein are useful for preventing and treating STDs. The terms “prevent,” “prevented” and “preventing” as used herein, refer to inhibiting completely or partially the onset of an STD, as well as, inhibiting an increase in the severity of an existing STD. The terms “treat,” “treated” and “treating” as used herein refer to decreasing the severity of an existing STD, as well as, in some cases, completely eliminating the STD. Thus, the term “prevention” embraces the use of the compounds of the invention for inhibiting the development of an STD before it begins. The term “treatment” embraces the use of the compounds of the invention for treating a subject in which an STD has already formed in order to slow or inhibit altogether the progression of the STD. The term “treatment” also embraces decreasing the severity of the disease, of disease related symptoms or of disease-related conditions, as described herein.

STDs intended to be prevented or treated by the methods and compositions of the invention include gonorrhoeae, syphilis, chlamydia, HPV (causing genital warts), AIDS/HIV, hepatitis, herpes simplex viruses I and II, trichomonas, candida, and chancroid, but are not so limited. Other STDs intended to be prevented or treated by the methods and compositions provided herein are scabies and pubic lice infections.

Sexually transmitted pathogens are generally bacterial, viral, parasitic or fungal in nature. Organisms that cause STDs include bacteria such as Neisseria gonorrhoeae, Chlamydia trachomatis, Treponema pallidum, Haemophilus ducreyi, Condyloma acuminata, Calymmatobacterium granulomatis and Ureaplasma urealyticum, viruses such as Human immunodeficiency viruses (HIV-1 and HIV-2), Human T lymphotropic virus type I (HTLV-I), Herpes simplex virus type 2 (HSV-2), Human papilloma virus (multiple types), Hepatitis B virus, Cytomegalovirus and Molluscum contagiosum virus, parasites such as Trichomonas vaginalis and Phthirus pubis, and fungi such as Candida albicans.

Other infections are known to be sexually transmitted, even if sexual transmission is not their predominant mode of transmission. This latter category includes infections caused by bacteria such as Mycoplasma hominis, Gardnerella vaginalis and Group B streptococcus, viruses such as Human T lymphotrophic virus type II (HTLV-II), Hepatitis C and D viruses, Herpes simplex virus type I (HSV-1) and Epstein-Barr virus (EBV), and parasites such as Sarcoptes scabiei.

The invention also intends to embrace STDs which are transmitted by sexual contact involving oral-fecal exposure. These STDs are caused by bacteria such as Shigella spp. and Campylobacter spp., viruses such as Hepatitis A virus and parasites such as Giardia lamblia and Entamoeba histolytica.

Many pathogen infections are not commonly sexually transmitted. The invention, in one aspect, does not intend to prevent or treat infections which are not known or have not been reported to be transmitted via sexual intercourse or through contact with bodily fluids. These infections are those which can be transmitted through a third party organism (e.g., insect) or through an agent other than bodily fluid (e.g., food). In a related aspect, however, the invention intends to prevent and treat these latter infections provided the active agents (e.g., nucleic acids and anti-STD agents) are administered in a manner which is directed solely to the sexual transmission of these pathogens. As an example, some of these infections may be treated or prevented via local delivery of the active agents to affected areas or suspected areas of transmission. Examples of such local delivery include, but are not limited to, vaginal, penile, anal or oral areas.

The active agents of the invention can be formulated or presented with some forms of birth control agents including but not limited to male and female hormonal contraceptives such as birth control pills, birth control implants or transdermal patches, and spermicides and spermicidal foams, or birth control devices including but not limited to condoms (male and female), intra-uterine devices, intra-vaginal devices, cervical caps, and contraceptive sponges. In one embodiment, the nucleic acids of the invention are administered with particular birth control devices in order to prevent infection during or following the implantation of such devices. For example, insertion of an intra-uterine device can lead to tears in the vaginal mucosa and the nucleic acids of the invention can prevent infection from developing due to such tearing.

In a further embodiment, the invention intends to treat some STD, provided that only a subset of the nucleic acids disclosed herein is used in the treatment and prevention therapies. As an example, infections caused by HIV and Herpes viruses, and the fungus Candida spp., when transmitted either by bodily or bodily fluid contact (such as in sexual activity) are preferably treated with non-CpG motif nucleic acids, and/or are preferably administered in a route other than a transdermal patch, injection or orally. Non-CpG nucleic acids, as used herein, are nucleic acids which do not contain a CpG motif (i.e., 5′ X₁X₂CGX₃X₄ 3′) as described herein.

In embodiments directed at the prevention and treatment of infections which have been sexually transmitted and in which local, preferably mucosal administration, is recommended, the nucleic acid may be one other than a CpG nucleic acid. The invention in some embodiments does not prevent or treat human STDs caused by HIV-1, HIV-2, HIV-3, HTLV-1, HTLV-II, HTLV-III, hepatitis A virus, hepatitis B virus, herpes simplex virus (HSV) 1 and 2, papilloma virus, Neisseria gonorrhoeae, Campylobacter sp., cytomegalovirus (CMV), Treponema pallidum, Chlamydia trachomatis and Candida albicans using local mucosal administration of CpG nucleic acids particularly when the subject is also exposed to the agent. However, the invention does provide compositions and kits for the administration of CpG and other nucleic acids to certain mucosal regions intended for use in the prevention or treatment of STDs including those listed immediately above. Examples of such compositions and kits include a birth control device or agent, a feminine sanitary product such as a douche, sanitary pad or, preferably a tampon, a vaginal or an anal suppository, or an enema, all of which may provide a nucleic acid and/or an anti-STD agent, and all of which may be provided as sustained release compositions (e.g., in a sustained release device).

In some embodiments, the nucleic acids alone are used to prevent and/or treat the infection. As an example, the subject may or may not be exposed either actively or passively to an antigen from an infectious agent at the time of the nucleic acid administration. Accordingly, the compositions provided herein may or may not comprise an antigen from the infectious agent. As used herein, an “antigen from the infectious agent” is the infectious agent or a fragment thereof (e.g., protein, carbohydrate, lipid, etc.) that the immune system recognizes as foreign (particularly when used in combination with the nucleic acids of the invention) and to which an antigen specific immune response can be mounted. Compositions that do not contain an antigen from an infectious agent are referred to herein as “non-vaccine compositions”.

The invention provides, in one aspect, a method for preventing or treating an STD in a subject in need thereof by administering a nucleic acid in an effective amount to prevent or treat the STD.

A “subject” a used herein is a human or non-human vertebrate animal including but not limited to dog, cat, rabbit, horse, cow, goat, sheep, pig, chicken, primate (e.g., monkey), rat, mouse and aquaculture species such as fish. In preferred embodiments, the subject is a human. The human subject may be one who engages in heterosexual, homosexual or bisexual activity. In some embodiments, the subject may not be engaged in sexual activity and may have acquired the STD through contact with the bodily fluid of an infected subject. Generally, a human subject will acquire an STD from an infected human subject.

A “subject in need thereof” may be a subject who is at risk of developing an STD or one who has an STD (i.e., a subject having an STD).

The nucleic acids are useful in some aspects as a prophylactic for the prevention of an STD in a subject at risk of developing an STD. A “subject at risk of developing an STD”, as used herein, is a subject who has any risk of developing an STD either by contact with an infected subject or by contact with a bodily fluid from an infected subject. For instance, a subject at risk is one who has or who will have a sexual partner who is infected with an STD-causing pathogen. Subjects at risk also include those who engage in unprotected sexual activity such as having sex, either oral, anal or vaginal, without a condom (i.e., male or female condom), regardless of whether they or their partners are aware of the existing infection. Subjects who have multiple sexual partners (e.g., prostitutes or those who frequent prostitutes) or who have even one sexual partner who in turn has multiple sexual partners are also considered to be at risk. Other subjects at risk of developing an STD are subjects who engage in other forms of high risk transmission behavior such as sharing of hypodermic needles. Subjects receiving blood products may also be considered to be at risk, particularly if the surveillance of the blood supply system is lax. An example of this latter category of subject is a subject in sub-Saharan African countries which have a blood supply system which is partially or completely contaminated with STD-causing pathogens (e.g., HIV). A subject at risk may also be one who is planning to travel to an area in which one or more STD-causing pathogens are common, particularly if it is known that such pathogens are present in the blood supply system of the area. Another subject at risk is one who has an occupation which involves potential contact with a bodily fluid of another. Examples of this latter category include, but are not limited to, nurses, doctors, dentists, and rescue personnel such as ambulance attendants, paramedics, fire-fighters, and police officers. Subjects at risk also include fetuses and newborns born to mothers who are infected with an STD-causing pathogen.

All of the afore-mentioned activities that are associated with the transmission of an STD causing pathogen are also referred to herein as “high risk activities”. The nucleic acid and potentially other prophylactic or therapeutic agents to be used in conjunction may be administered before, or during, or following the time which the subject is engaged in the high risk activity. A subject who is administered a nucleic acid before engaging in sexual activity, for example, may receive the nucleic acid at least one month, at least one week, at least 48 hours, at least 24 hours, at least 12 hours, at least 6 hours, at least 4 hours, at least 2 hours (or any time therebetween as if such time was explicitly recited herein) prior to having sex. Preferably, the time of administration prior to engagement in the high risk activity is a time sufficient to activate the immune system so that it is active while the infectious agent is present in the body of the subject. A subject who is administered the nucleic acid following engagement in the high risk activity may receive it within 2 hours, within 4 hours, within 6 hours, within 12 hours, within 24 hours, within 48 hours, or within 3, 4, 5, 6, 7, 14, 28 days or longer (or any time therebetween as if such time was explicitly recited herein) after engaging in the high risk activity.

The efficiency of transmission of STD-causing pathogens is dependent upon the particular pathogen. Thus, subjects having sexual contact with another who is infected with N. gonorrhoeae are more likely to become infected than subjects having sexual contact with another who is infected with HIV. Similarly, the period of infectivity will differ depending upon the pathogen. Most STDs are more easily transmitted from males to females, and thus females are disproportionately affected by STDs, as are their children, especially those in utero.

In addition to prophylaxis of STDs, the invention also encompasses treatment of a subject having an STD. A “subject having an STD” is a subject that has been infected with an STD-causing pathogen, and in some instances has symptoms that are associated with STDs. STDs may manifest themselves through symptoms such as overt genital discharge, genital lesions and pain, itching in the genital region, the urge to urinate frequently, burning sensation during urination, pain and discomfort in the rectal area, a sore throat (in the case of N. gonorrhoeae transmitted through oral sex), tenemus (a persistent urge to empty the bowels), inflammation of genital tissues, bleeding between menstrual periods and pain during sexual activity.

Genital warts may manifest themselves as small, flat, flesh-colored bumps or tiny, cauliflower-like bumps usually measuring between 1-2 mm in diameter. Syphilis causes open ulcers in the anal and/or oral area (primary syphilis), rashes on the palms of the hands and soles of the feet and/or white patches in the mouth, fever, headaches, (secondary syphilis) and tumors of the mouth, nose, tongue, bone and skin, joint pain, vomiting and abdominal pain, paralysis, loss of sensation, blindness, deterioration of intellectual function and impotence (tertiary syphilis). Chancroid may manifest itself as painful and tender sores in the region of the lips, mouth, throat, anus, tongue, vagina or penis, with swollen glands near the affected area. Pubic lice are usually associated with a delayed itching sensation and redness in the genital area. Hepatitis is usually associated with nausea, vomiting, fatigue, headache, fever, jaundice with yellow color seen in the eyes, skin and bodily fluids, and light colored stool. Herpes may cause small blisters or sores in the mouth or genital areas.

Some subjects infected with STD-causing pathogens however may be asymptomatic. Female subjects infected with an STD-causing pathogen are generally more likely to be asymptomatic than males similarly infected. If the subject has participated in unprotected sexual activity, has multiple sexual partners, or is aware that one or more sexual partners are infected, and the subject is still asymptomatic, then the subject can be definitively diagnosed using clinical tests.

Several methods for diagnosing an STD are known in the art and may be used when practicing the invention. Generally, diagnosis is made by trained personnel including a nurse, a nurse practitioner or a physician. Such methods include taking a full sexual history of the subject, physical examination of the genital area, mouth, throat and palpitation of the liver (hepatitis), microscopic analysis of wet mounts of discharge samples (especially for female subjects), Gram's stain of smear or swab samples (especially for male subjects), pap smear, urethral or cervical swabs, microbiological culture tests, antigen detection tests, antibody detection assays (e.g., HIV Antibody Test, MHA-T for antibody to T pallidum (syphilis)), urine tests, rapid slide coagglutination tests, serologic tests including enzyme tests for proper liver functioning (hepatitis), Meridian Diagnostics Premiere® tests for HSV-1 and HSV-2, and Diagnology's POCkit®HSV-2 Rapid Test, and PCR tests for detecting particular pathogen-specific nucleic acids. In some microscopic analyses, a definitive diagnosis is made if neutrophil numbers are elevated, or if microorganisms are clearly visible.

The nucleic acids of the invention are also useful for preventing and/or treating yeast infections in subjects at risk of having or developing or subjects having a yeast infection. The yeast infection may exist locally such as in the genital area, or in the mouth, but in other cases it may be disseminated throughout the body (including the mouth, genital area, esophagus, and skin). Subjects at risk of having or developing a yeast infection include females generally, and especially females that have previously had a yeast infection, subjects who have been administered antibiotics (e.g., tetracycline) and diaper-wearing children. Also at risk of having yeast infections are females who are taking estrogen-containing birth control agents, and females that are pregnant. Subjects who are immunocompromised (e.g., subjects who are HIV⁺ and who are experiencing AIDS related symptoms), as well as those who are diabetic are also at risk of having a yeast infection. Yeast infections are known to afflict infants, toddlers and children (herein after referred to collectively as “children”) of both sexes. Symptoms associated with a yeast infection include vaginal and labial itching, abnormal vaginal discharge, pain during sexual intercourse and/or during urination and rashes (e.g., a penile rash). The method of prevention and/or treatment includes local administration of the nucleic acids of the invention to the area where the yeast infection is likely to exist or where it does exist. Vaginal yeast infections (e.g., candidiasis or monilial vaginitis) are often caused by the fungus Candida albicans. In some embodiments, the yeast infection is not sexually transmitted, and it is rather due to an imbalance in, for example, the subject's genital environment, leading to an increase in the growth of an already and perhaps normally present fungus.

The compounds useful according to the invention are nucleic acids. The nucleic acids may be double-stranded or single-stranded. Generally, double-stranded molecules may be more stable in vivo, while single-stranded molecules may have increased activity. The terms “nucleic acid” and “oligonucleotide” refer to multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. The terms “nucleic acid” and “oligonucleotide” also encompass nucleic acids or oligonucleotides with substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus modified nucleic acids may include a 2′-O-alkylated ribose group. In addition, modified nucleic acids may include sugars such as arabinose instead of ribose. Thus the nucleic acids may be heterogeneous in backbone composition thereby containing any possible combination of polymer units linked together such as peptide- nucleic acids (which have amino acid backbone with nucleic acid bases). In some embodiments, the nucleic acids are homogeneous in backbone composition.

Nucleic acids also include substituted purines and pyrimidines such as C-5 propyne modified bases (Wagner et al., Nature Biotechnology 14:840-844, 1996). Purines and pyrimidines include but are not limited to adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties.

The nucleic acid is a linked polymer of bases or nucleotides. As used herein with respect to linked units of a nucleic acid, linked or linkage means two entities are bound to one another by any physicochemical means. Any linkage known to those of ordinary skill in the art, covalent or non-covalent, is embraced. Such linkages are well known to those of ordinary skill in the art. Natural linkages, which are those ordinarily found in nature connecting the individual units of a nucleic acid, are most common. The individual units of a nucleic acid may be linked, however, by synthetic or modified linkages.

Whenever a nucleic acid is represented by a sequence of letters it will be understood that the nucleotides are in 5′→3′ order from left to right and that A denotes adenosine, C denotes cytosine, G denotes guanosine, T denotes thymidine, and U denotes uracil unless otherwise noted.

Nucleic acid molecules useful according to the invention can be obtained from natural nucleic acid sources (e.g. genomic nuclear or mitochondrial DNA or cDNA), or are synthetic (e.g. produced by oligonucleotide synthesis). Nucleic acids isolated from existing nucleic acid sources are referred to herein as native, natural, or isolated nucleic acids. The nucleic acids useful according to the invention may be isolated from any source, including eukaryotic sources, prokaryotic sources, nuclear DNA, mitochondrial DNA, etc. Thus, the term nucleic acid encompasses both synthetic and isolated nucleic acids. The term isolated as used herein refers to a nucleic acid which is substantially free of other nucleic acids, proteins, lipids, carbohydrates or other materials with which it is naturally associated. The nucleic acids can be produced on a large scale in plasmids, (see Sambrook, T., et al., Molecular Cloning: A Laboratory Manual, Cold Spring Harbor laboratory Press, New York, 1989) and separated into smaller pieces or administered whole. After being administered to a subject the plasmid can be degraded into oligonucleotides. One skilled in the art can purify viral, bacterial, eukaryotic, etc. nucleic acids using standard techniques, such as those employing restriction enzymes, exonucleases or endonucleases.

For use in the instant invention, the nucleic acids can be synthesized de novo using any of a number of procedures well known in the art. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et. al., Nucl. Acid. Res. 14:5399-5407, 1986, ; Garegg et al., Tet. Let. 27:4055-4058, 1986, Gaffney et al., Tet. Let. 29:2619-2622, 1988). These chemistries can be performed by a variety of automated oligonucleotide synthesizers available in the market.

In some embodiments, the nucleic acids useful according to the invention are immunostimulatory nucleic acids. An immunostimulatory nucleic acid is any nucleic acid, as described above, which is capable of modulating an immune response. A nucleic acid which modulates an immune response is one which produces any form of immune stimulation, including, but not limited to, induction of cytokines, B cell activation, T cell activation, monocyte activation. Accordingly, the immune responses induced by the nucleic acids of the invention can be either or both innate and adaptive immune responses. Immunostimulatory nucleic acids include, but are not limited to, CpG nucleic acids, methylated CpG nucleic acids, T-rich nucleic acids, poly G nucleic acids, and nucleic acids having phosphate modified backbones, such as phosphorothioate backbones.

A CpG nucleic acid or a CpG immunostimulatory nucleic acid as used herein is a nucleic acid containing at least one unmethylated CpG dinucleotide (cytosine-guanine dinucleotide sequence, i.e. CpG DNA or DNA containing an unmethylated 5′ cytosine followed by 3′ guanosine and linked by a phosphate bond) and activates a component of the immune system. The entire CpG nucleic acid can be unmethylated or portions may be unmethylated but at least the C of the 5′ CG 3′ must be unmethylated.

Methylated CpG nucleic acids are also immunostimulatory and useful for the purposes of the methods of the invention. A methylated CpG nucleic acid is a nucleic acid containing at least one CG dinucleotide in which the C of the CG is methylated and which does not include any unmethylated CG dinucleotides.

In one embodiment, the invention provides a CpG nucleic acid or a methylated CpG nucleic acid represented by at least the formula:

5′N₁X₁CGX₂N₂3′

wherein X₁ and X₂ are nucleotides and N is any nucleotide and N₁ and N₂ are nucleic acid sequences composed of from about 0-25 N's each. In some embodiments, X₁ is adenine, guanine, or thymine and X₂ is cytosine, adenine, or thymine. In other embodiments, X₁ is cytosine and/or X₂ is guanine.

In other embodiments, the CpG nucleic acid or methylated CpG nucleic acid is represented by at least the formula:

5′N₁X₁X₂CGX₃X₄N₂3′

wherein X₁, X₂, X₃, and X₄ are nucleotides. In some embodiments, X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA; N is any nucleotide and N₁ and N₂ are nucleic acid sequences composed of from about 0-25 N's each. In some embodiments, X₁X₂ are GpA or GpT and X₃X₄ are TpT. In other embodiments, X₁ or X₂ or both are purines and X₃ or X₄ or both are pyrimidines or X₁X₂ are GpA and X₃ or X₄ or both are pyrimidines.

In some embodiments, N₁ and N₂ of the nucleic acid do not contain a CCGG or CGCG quadmer or more than one CCG or CGG trimer. The effect of a CCGG or CGCG quadmer or more than one CCG or CGG trimer depends in part on the status of the nucleic acid backbone. For instance, if the nucleic acid has a phosphodiester backbone or a chimeric backbone the inclusion of these sequences in the nucleic acid will only have minimal if any affect on the biological activity of the nucleic acid. If the backbone is completely phosphorothioate or significantly phosphorothioate then the inclusion of these sequences may have more influence on the biological activity or the kinetics of the biological activity, but compounds containing these sequences are still useful. In another embodiment, the CpG nucleic acid or the methylated CpG nucleic acid has the sequence 5′TCN₁TX₁X₂CGX₃X₄3′. Examples of CpG nucleic acids include but are not limited to those listed described in U.S. Non-Provisional Patent Application Serial No. 09/669,187, filed Sep. 25, 2000.

A T-rich nucleic acid or T-rich immunostimulatory nucleic acid is a nucleic acid which includes at least one poly T sequence and/or which has a nucleotide composition of greater than 25% T nucleotide residues and which activates a component of the immune system. A nucleic acid having a poly-T sequence includes at least four Ts in a row, such as 5′TTTT3′. Preferably the T-rich nucleic acid includes more than one poly T sequence. In preferred embodiments, the T-rich nucleic acid may have 2, 3, 4, etc. poly T sequences. Other T-rich nucleic acids have a nucleotide composition of greater than 25% T nucleotide residues, but do not necessarily include a poly T sequence. In these T-rich nucleic acids the T nucleotide resides may be separated from one another by other types of nucleotide residues, i.e., G, C, and A. In some embodiments, the T-rich nucleic acids have a nucleotide composition of greater than 30%, 40%, 50%, 60%, 70%, 80%, 90%, and 99%, T nucleotide residues and every integer % in between. Preferably the T-rich nucleic acids have at least one poly T sequence and a nucleotide composition of greater than 25% T nucleotide residues.

In one embodiment, the T-rich nucleic acid is represented by at least the formula:

5′X₁X₂TTTTX₃X₄3′

wherein X₁, X₂,X₃, and X₄ are nucleotides. In one embodiment, X₁X₂ is TT and/or X₃X₄ is TT. In another embodiment, X₁X₂ are any one of the following nucleotides TA, TG, TC, AT, AA, AG, AC, CT, CC, CA, CG, GT, GG, GA, and GC; and X₃X₄ are any one of the following nucleotides TA, TG, TC, AT, AA, AG, AC, CT, CC, CA, CG, GT, GG, GA, and GC.

In some embodiments, it is preferred that the T-rich nucleic acid does not contain poly C (CCCC), poly A (AAAA), poly G (GGGG), CpG motifs, or multiple GGs. In other embodiments, the T-rich nucleic acid includes these motifs. Thus in some embodiments, of the invention the T-rich nucleic acids include CpG dinucleotides and in other embodiments, the T-rich nucleic acids are free of CpG dinucleotides. The CpG dinucleotides may be methylated or unmethylated.

Examples of T rich nucleic acids that are free of CpG nucleic acids include but are not limited to those described in U.S. Non-Provisional Patent Application Serial No. 09/669,187, filed Sep. 25, 2000. This application also describes examples of T rich nucleic acids that include CpG nucleic acids.

Poly G containing nucleic acids are also immunostimulatory. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J Biochem. 116, 991-994 also describe the immunostimulatory properties of poly G nucleic acids.

Poly G nucleic acids preferably are nucleic acids having the following formulas:

5′X₁X₂GGGX₃X₄ 3′

wherein X₁, X₂, X₃, and X₄ are nucleotides. In preferred embodiments, at least one of X₃ and X₄ are a G. In other embodiments, both of X₃ and X₄ are a G. In yet other embodiments, the preferred formula is 5′ GGGNGGG 3′, or 5′ GGGNGGGNGGG 3′ wherein N represents between 0 and 20 nucleotides.

In other embodiments the poly G nucleic acid is free of unmethylated CG dinucleotides, such as, for example, the nucleic acids listed in U.S. Non-Provisional Patent Application Serial No. 09/669,187, filed Sep. 25, 2000. This latter application also provides examples of poly G nucleic acids that include at least one unmethylated CG dinucleotide.

U.S. Non-Provisional Patent Application Serial No. 09/669,187, filed Sep. 25, 2000 lists a number of nucleic acids that can be used in the invention. The base designations other than a, c, g and t (or u) (all of which are known in the art) are as follows: i intends inosine; n intends a, c, g, or t, other; d intends a, g, t or u; h intends a, c or t (or u); b intends c, g or t (or u), however if “b” is single and is listed on 5′ or 3′ end of oligonucleotide, then “b” indicates a biotin moiety attached to that end of the oligonucleotide; q intends 5-methyl-cytosine; m intends a or c; s intends c or g; x, if single and is listed on 5′ or 3′ end of oligonucleotide, intends a biotin moiety attached to that end of the oligonucleotide; z intends 5-methyl-cytidine; and f intends at least one FITC moiety attached to 5′ or 3′ end of oligonucleotide.

The backbone modifications listed are as follows: Backbone modifications are abbreviated as follows: S intends phosphorothioate; O intends phosphodiester; SOS intends phosphorothioate and phosphodiester chimeric with phosphodiester in middle; SO intends phosphorothioate and phosphodiester chimeric with phosphodiester on 3′ end; OS intends phosphorothioate and phosphodiester chimeric with phosphodiester on 5′ end; S2 intends phosphorodithioate; S2O intends phosphorodithioate and phosphodiester chimeric with phosphodiester on 3′ end; OS2 intends phosphorodithioate and phosphodiester chimeric with phosphodiester on 5′ end; X intends any of the above; and p-ethoxy intends p-ethoxy backbone as in e.g., U.S. Pat. No. 6,015,886. In some instances, the nucleic acid may also have a peptide backbone such as for example as in peptide nucleic acids which are known in the art.

Nucleic acids having modified backbones, such as phosphorothioate backbones, also fall within the class of immunostimulatory nucleic acids. U.S. Pat. Nos. 5,723,335 and 5,663,153 issued to Hutcherson, et al. and related PCT publication WO95/26204 describe immune stimulation using phosphorothioate oligonucleotide analogues. These patents describe the ability of the phosphorothioate backbone to stimulate an immune response in a non-sequence specific manner.

The immunostimulatory nucleic acid may be any size of at least 2 nucleotides but in some embodiments, are in the range of between 6 and 100 or in some embodiments, between 8 and 35 nucleotides in size. Immunostimulatory nucleic acids can be produced on a large scale in plasmids. These may be administered in plasmid form or alternatively they can be degraded into oligonucleotides.

Palindromic sequence shall mean an inverted repeat (i.e. a sequence such as ABCDEE′D′C′B′A′ in which A and A′ are bases capable of forming the usual Watson-Crick base pairs and which includes at least 6 nucleotides in the palindrome. In vivo, such sequences may form double-stranded structures. In one embodiment, the nucleic acid contains a palindromic sequence. In some embodiments, when the nucleic acid is a CpG nucleic acid, a palindromic sequence used in this context refers to a palindrome in which the CpG is part of the palindrome, and optionally is the center of the palindrome. In another embodiment, the nucleic acid is free of a palindrome. A nucleic acid that is free of a palindrome does not have any regions of 6 nucleotides or greater in length which are palindromic. A nucleic acid that is free of a palindrome can include a region of less than 6 nucleotides which are palindromic.

A stabilized nucleic acid molecule shall mean a nucleic acid molecule that is relatively resistant to in vivo degradation (e.g. via an exo- or endo-nuclease). Stabilization can be a function of length or secondary structure. Nucleic acids that are tens to hundreds of kbs long are relatively resistant to in vivo degradation. For shorter nucleic acids, secondary structure can stabilize and increase their effect. For example, if the 3′ end of an oligonucleotide has self-complementarity to an upstream region, so that it can fold back and form a sort of stem loop structure, then the oligonucleotide becomes stabilized and therefore exhibits more activity.

Some stabilized oligonucleotides of the instant invention have a modified backbone. It has been demonstrated that modification of the oligonucleotide backbone provides enhanced activity of the nucleic acids when administered in vivo. Nucleic acids, including at least two phosphorothioate linkages at the 5′ end of the oligonucleotide and multiple phosphorothioate linkages at the 3′ end, preferably 5, may provide maximal activity and protect the oligonucleotide from degradation by intracellular exo- and endo-nucleases. Other modified oligonucleotides include phosphodiester modified oligonucleotide, combinations of phosphodiester and phosphorothioate oligonucleotide, methylphosphonate, methylphosphorothioate, phosphorodithioate, and combinations thereof. Each of these combinations and their particular effects on immune cells is discussed in more detail in PCT Published Patent Applications claiming priority to U.S. Ser. Nos. 08/738,652 and 08/960,774, filed on Oct. 30, 1996 and Oct. 30, 1997 respectively, the entire contents of which is hereby incorporated by reference. It is believed that these modified oligonucleotides may show more stimulatory activity due to enhanced nuclease resistance, increased cellular uptake, increased protein binding, and/or altered intracellular localization.

Both phosphorothioate and phosphodiester nucleic acids are active in immune cells. Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

For use in vivo, nucleic acids are preferably relatively resistant to degradation (e.g., via endo-and exo-nucleases). Secondary structures, such as stem loops, can stabilize nucleic acids against degradation. Alternatively, nucleic acid stabilization can be accomplished via phosphate backbone modifications. One type of stabilized nucleic acid has at least a partial phosphorothioate modified backbone. Phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990). Other sources of nucleic acids useful according to the invention include standard viral and bacterial vectors, many of which are commercially available. In its broadest sense, a vector is any nucleic acid material which is ordinarily used to deliver and facilitate the transfer of nucleic acids to cells. The vector as used herein may be an empty vector or a vector carrying a gene which can be expressed. In the case when the vector is carrying a gene the vector generally transports the gene to the target cells with reduced degradation relative to the extent of degradation that would result in the absence of the vector. In this case the vector optionally includes gene expression sequences to enhance expression of the gene in target cells such as immune cells, but it is not required that the gene be expressed in the cell.

In general, vectors include, but are not limited to, plasmids, phagemids, viruses, other vehicles derived from viral or bacterial sources. Viral vectors are one type of vector and include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Some viral vectors are based on non-cytopathic eukaryotic viruses in which non-essential genes have been replaced with a nucleic acid to be delivered. Non-cytopathic viruses include retroviruses, the life cycle of which involves reverse transcription of genomic viral RNA into DNA.

Standard protocols for producing empty vectors or vectors carrying genes (including the steps of incorporation of exogenous genetic material into a plasmid, transfection of a packaging cell lined with plasmid, production of recombinant retroviruses by the packaging cell line, collection of viral particles from tissue culture media, and/or infection of the target cells with viral particles) are provided in Kriegler, M., Gene Transfer and Expression, A Laboratory Manual, W. H. Freeman C. O., New York (1990) and Murry, E. J. Ed. Methods in Molecular Biology, vol. 7, Humana Press, Inc., Cliffton, N.J. (1991). Other vectors include plasmid vectors. Plasmid vectors have been extensively described in the art and are well-known to those of skill in the art. See e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. Some plasmids, however, having a promoter compatible with the host cell, can express a peptide from a gene operatively encoded within the plasmid. Some commonly used plasmids include pBR322, pUC18, pUC19, pcDNA3.1, SV40, and pBlueScript. Other plasmids are well-known to those of ordinary skill in the art. Additionally, plasmids may be custom designed using restriction enzymes and ligation reactions to remove and add specific fragments of DNA.

It has recently been discovered that plasmids (empty or gene carrying) can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e.g. dendritic cells, probably by passing through the gut barrier. High levels of immune protection have been established using this methodology. Such methods of delivery are useful for the aspects of the invention utilizing systemic delivery of nucleic acid.

The compounds of the invention may be administered alone or in combination with an anti-STD agent. An anti-STD agent, as used herein, refers to any compound which is useful for preventing or treating STDs. These compounds include, for instance, any of the compounds described herein as well as any other compounds which have been suggested to be useful for the treatment of STDs, including, but not limited to, antibodies to STD-causing pathogens and anti-sense therapy directed to STD-causing pathogens.

Many types of drugs have been proposed and developed for the treatment of STDs. Important anti-STD agents include, but are not limited to, anti-bacterial agents, anti-viral agents, anti-parasite agents and anti-fungal agents.

Anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

The anti-bacterial agent may be an antibiotic, such as a broad spectrum antibiotic, a narrow spectrum antibiotic, or a limited spectrum antibiotic. Examples of anti-bacterial agents include, but are not limited to, natural penicillins, semi-synthetic penicillins, clavulanic acid, cephalolsporins, bacitracin, ampicillin, carbenicillin, oxacillin, azlocillin, mezlocillin, piperacillin, methicillin, dicloxacillin, nafcillin, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, carbapenems, imipenems, monobactems, euztreonam, vancomycin, polymyxin, amphotericin B, nystatin, imidazoles, clotrimazole, miconazole, ketoconazole, itraconazole, fluconazole, rifampins, ethambutol, tetracyclines, chloramphenicol, macrolides, aminoglycosides, streptomycin, kanamycin, tobramycin, amikacin, gentamicin, tetracycline, minocycline, doxycycline, chlortetracycline, erythromycin, roxithromycin, clarithromycin, oleandomycin, azithromycin, chloramphenicol, quinolones, co-trimoxazole, norfloxacin, ciprofloxacin, enoxacin, nalidixic acid, temafloxacin, sulfonamides, gantrisin, and trimethoprim.

Other anti-bacterial agents include Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate ; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride ; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel ; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin ; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; and Zorbamycin.

In some important embodiments, the anti-bacterial agent is selected from the group consisting of ampicillin/amoxicillin, amoxicillin/clarithromycin combination, azithromycin (C. trachomatis), cefixime (C. trachomatis), cefotetan, cefoxitin, ceftriaxone, ciprofloxacin (C. trachomatis), clarithromycin, clindamycin (T. pallidum, G. vaginalis), doxycycline (C. trachomatis), gentamicine/tobramycin, metronidazole (T. pallidum, G. vaginalis, T vaginalis), naphthyridine carboxylic acid antibacterial compounds, ofloxacin (C. trachomatis), spectinomycin (C. trachomatis), tetracycline HCl, trovafloxacin (C. trachomatis).

Some strains of STD-causing pathogens have developed antibiotic resistance. Hence the nucleic acids of the invention are useful for circumventing this resistance by providing an alternate mechanism for treating these infections. In addition, treatment of STDs with some forms of antibiotics sometimes leads to the development of a yeast infection in females owing to the overgrowth of endogenous vaginal yeast. Treatment of STDs with nucleic acids can be useful in maintaining a balance in the vaginal flora and thereby reduce the chance of a yeast infection.

Anti-viral agents include immunoglobulins, amantadine, interferon, nucleoside analogues, and protease inhibitors. In some embodiments, relating to the treatment of hepatitis, interferon and lamivudine are the anti-virals of choice. Alpha-interferon, trichloroacetic acid, podophyllin, imiquimod, and fluorouracil are useful anti-viral agent in the prevention and treatment genital warts, particularly those caused by human papilloma virus (HPV), Acyclovir (Zovirax®), valacyclovir (Valtrex®) and famciclovir (Famvir®) are all particularly useful anti-viral agents in the prevention and treatment of herpes.

The anti-viral agent may be further selected from the group consisting of Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-parasite agents are well known in the art and generally commercially available. Examples of parasiticides useful for human administration include, but are not limited to, albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HC1, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole.

Parasiticides used in non-human subjects include piperazine, diethylcarbamazine, thiabendazole, fenbendazole, albendazole, oxfendazole, oxibendazole, febantel, levamisole, pyrantel tartrate, pyrantel pamoate, dichlorvos, ivermectin, doramectic, milbemycin oxime, iprinomectin, moxidectin, N-butyl chloride, toluene, hygromycin B thiacetarsemide sodium, melarsomine, praziquantel, epsiprantel, benzimidazoles such as fenbendazole, albendazole, oxfendazole, clorsulon, albendazole, amprolium; decoquinate, lasalocid, monensin sulfadimethoxine; sulfamethazine, sulfaquinoxaline, metronidazole.

Parasiticides used in horses include mebendazole, oxfendazole, febantel, pyrantel, dichlorvos, trichlorfon, ivermectin, piperazine; for S. westeri: ivermectin, benzimiddazoles such as thiabendazole, cambendazole, oxibendazole and fenbendazole. Useful parasiticides in dogs include milbemycin oxine, ivermectin, pyrantel pamoate and the combination of ivermectin and pyrantel. The treatment of parasites in swine can include the use of levamisole, piperazine, pyrantel, thiabendazole, dichlorvos and fenbendazole. In sheep and goats anthelmintic agents include levamisole or ivermectin. Caparsolate has shown some efficacy in the treatment of D. immitis (heartworm) in cats.

Agents used in the prevention and treatment of protozoal diseases in poultry, particularly trichomoniasis, include protozoacides such as aminonitrothiazole, dimetridazole (Emtryl), nithiazide (Hepzide) and Enheptin.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream). In some important embodiments, the anti-fungal agent of choice, preferably in the prevention or treatment of Candida albicans infection may be selected from the group of amphoterizin B, miconazole, clotrimazole, 5-fluorocytosine, fluconazole, fluconazole, itraconazole and voriconazole.

Some examples of commercially-available anti-fungal agents are shown in Table 2.

TABLE 2

| Company | Brand Name | Generic Name | Indication | Mechanism of Action |
|---|---|---|---|---|
| PHARMACIA & | PNU 196443 | PNU 196443 | Anti Fungal | n/k |
| UPJOHN | | | | |
| Lilly | LY 303366 | Basiungin/ECB | Fungal Infections | Anti-fungal/cell wall |
| | | | | inhibitor, glucose |
| | | | | synthase inhibitor |
| Lilly | LY 303366 | Basiungin/ECB | Fungal Infections | Anti-fungal/cell wall |
| | | | | inhibitor, glucose |
| | | | | synthase inhibitor |
| Bayer | Canesten | Clotrimazole | Fungal Infections | Membrane integrity |
| | | | | destabilizer |
| Fujisawa | FK 463 | FK 463 | Fungal Infections | Membrane integrity |
| | | | | destabilizer |
| Mylan | Sertaconzaole | Sertaconzole | Fungal Infections | Membrane integrity |
| | | | | destabilizer |
| Genzyme | Chitinase | Chitinase | Fungal Infections, Systemic | Chitin Breakdown |
| Liposome | Abelcet | Amphotericin B, | Fungal Infections, Systemic | Membrane integrity |
| | | Liposomal | | destabilizer |
| Liposome | Abelcet | Amphotericin B, | Fungal Infections, Systemic | Membrane integrity |
| | | Liposomal | | destabilizer |
| Sequus | Amphotec | Amphotericin B, | Fungal Infections, Systemic | Membrane integrity |
| | | Liposomal | | destabilizer |
| Sequus | Amphotec | Amphotericin B, | Fungal Infections, Systemic | Membrane integrity |
| | | Liposomal | | destabilizer |
| Bayer | BAY 38-9502 | BAY 38-9502 | Fungal Infections, Systemic | Membrane integrity |
| | | | | destabilizer |
| Pfizer | Diflucan | Fluconazole | Fungal Infections, Systemic | Membrane integrity |
| | | | | destabilizer |
| Pfizer | Diflucan | Fluconazole | Fungal Infections, Systemic | Membrane integrity |
| | | | | destabilizer |
| Johnson & Johnson | Sporanox | Itraconazole | Fungal Infections, Systemic | Membrane integrity |
| | | | | destabilizer |
| Johnson & Johnson | Sporanox | Itraconazole | Fungal Infections, Systemic | Membrane integrity |
| | | | | destabilizer |
| Sepracor | Itraconzole (2R, 4S) | Itraconzole (2R, 4S) | Fungal Infections, Systemic | Membrane integrity |
| | | | | destabilizer |
| Johnson & Johnson | Nizoral | Ketoconazole | Fungal Infections, Systemic | Membrane integrity |
| | | | | destabilizer |
| Johnson & Johnson | Nizoral | Ketoconazole | Fungal Infections, Systemic | Membrane integrity |
| | | | | destabilizer |
| Johnson & Johnson | Monistat | Miconazole | Fungal Infections, Systemic | Membrane integrity |
| | | | | destabilizer |
| Johnson & Johnson | Monistat | Miconazole | Fungal Infections, Systemic | Membrane integrity |
| | | | | destabilizer |
| Merck | MK 991 | MK 991 | Fungal Infections, Systemic | Membrane integrity |
| | | | | destabilizer |
| Merck | MK 991 | MK 991 | Fungal Infections, Systemic | Membrane integrity |
| | | | | destabilizer |
| Bristol Myers Sq'b | Pradimicin | Pradimicin | Fungal Infections, Systemic | Membrane integrity |
| | | | | destabilizer |
| Pfizer | UK-292, 663 | UK-292, 663 | Fungal Infections, Systemic | Membrane integrity |
| | | | | destabilizer |
| Pfizer | UK-292, 663 | UK-292, 663 | Fungal Infections, Systemic | Membrane integrity |
| | | | | destabilizer |
| Pfizer | Voriconazole | Voriconazole | Fungal Infections, Systemic | Membrane integrity |
| | | | | destabilizer |
| Pfizer | Voriconazole | Voriconazole | Fungal Infections, Systemic | Membrane integrity |
| | | | | destabilizer |
| Mylan | 501 Cream | 501 Cream | Inflammatory Fungal | Immunosuppression |
| | | | Conditions | |
| Mylan | Mentax | Butenafine | Nail Fungus | Membrane Integrity |
| | | | | Destabiliser |
| Schering Plough | Anti Fungal | Anti Fungal | Opportunistic Infections | Membrane Integrity |
| | | | | Destabiliser |
| Schering Plough | Anti Fungal | Anti Fungal | Opportunistic Infections | Membrane Integrity |
| | | | | Destabiliser |
| Alza | Mycelex Troche | Clotrimazole | Oral Thrush | Membrane Integrity |
| | | | | Stabliser |
| Novartis | Lamisil | Terbinafine | Systemic Fungal Infections, | Membrane Integrity |
| | | | Onychomycosis | Destabiliser |

If the STD is pubic lice or scabies mite, the anti-STD agent may be selected from the group consisting of Kwell (in the form of a lotion, shampoo, or cream), lindane and permethrin.

In still other embodiments, the nucleic acids of the invention may be administered to a subject having or at risk of having an STD along with a non-drug anti-STD therapy. A non-drug anti-STD therapy includes cryotherapy and laser therapy, both of which are useful in the treatment of genital warts.

Other therapeutic agents which can be administered with the nucleic acids of the invention, and optionally with the anti-STD agents, are mucosal adjuvants. In some embodiments, of the invention, particularly where a mucosal adjuvant is used, the nucleic acid is a non-CpG nucleic acid. Mucosal adjuvants are most preferably used when the nucleic acids are administered directly to a mucosal surface. The mucosal adjuvants useful according to the invention are non-oligonucleotide mucosal adjuvants. A “non-oligonucleotide mucosal adjuvant” as used herein is an adjuvant other than a CpG oligonucleotide that is capable of inducing a mucosal immune response in a subject when administered to a mucosal surface in conjunction with an antigen. Mucosal adjuvants include but are not limited to Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE1 12K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); andCTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler BD, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis)(Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worster, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

In a preferred embodiment, the nucleic acid and/or the anti-STD agent is provided together with a birth control agent such as male and female hormonal contraceptives such as a birth control pill, a hormonal implant, the morning after pill (i.e., high dose estrogen pill, e.g., RU486), or a spermicide in the form of a foam, gel, lotion, jelly, ointment or coating or a birth control device (e.g., a barrier method) such as an intra-uterine device (IUD), an intra-vaginal device (IVD), a diaphragm, a cervical cap, a sponge, a suppository, or a condom (male and female). An example of a spermicide is the compound nonoxynol-9 which is a nonionic detergent capable of lysing sperm. However, due to its detergent properties and the fact that it is most effective when administered to the vaginal mucosa directly or via a coated condom (male and female), nonoxynol-9 has a side effect of inducing vaginal and/or cervical irritation. As discussed below, administration of the nucleic acids of the invention together with nonoxynol-9 (or another adverse side-effect or dose limited anti-STD agent) may allow for lower doses of nonoxynol-9 (or other anti-STD agent) to be administered to the subject without loss of therapeutic value.

The nucleic acids may also be incorporated into birth control pills or pellets so that subjects using birth control pills would also receive nucleic acids for the prevention and/or treatment of STDs. As used herein, birth control pills or pellets embrace both male hormonal contraceptives and female hormonal contraceptives. Male hormonal contraceptives include but are not limited to oral gestogen with testosterone, 7alpha-methyl- 19-nortestosterone (MENT), and synthetic oral progestogen (desogestrel (DSG)). As an example, the nucleic acid may be incorporated into each pill of a one-month cycle or supply of pills, or may be provided in every second, third, fourth, fifth, sixth, seventh, tenth, twelfth, fifteenth, twenty-first, or twenty-eighth pill enclosed in the package, depending upon the number of pills in the supply. Examples of birth control pills and/or hormone formulations which could be so used according to the invention include Ortho-Novum 1/50, Norinyl 1/50, Ovcon 1/50, Ovral, Demulen, Norlestrin 2.5/50, Norlestrin 1/50, Ortho Novum 1/35, Norinyl 1+35, Modicon, Brevicon, Ovcon 35, Demulen 1/35, Loestrin 1.5/30, Loestrin 1/20, Nordette, Lo-Ovral, Ortho-Novum 10/11, Ortho-Novum 7/7/7, Tri-Norinyl, Triphasil, and Tri-Levein, Micronor, Nor Q. D., and Ovrette.

Similarly, the nucleic acids may be incorporated into sustained release devices intended for birth control. An example of such a device is the Norplant implant which is intended for birth control hormone release for months and, in some cases, years.

In another aspect, the invention is intended to prevent or treat STD-related conditions. STD-related conditions are conditions, disorders or diseases which result from an STD (i.e., they are secondary to the initial sexually transmitted infection). These include acute arthritis (N. gonorrhoeae (e.g., DGI), C. trachomatis (e.g., Reiter's syndrome), HBV, HIV), acute pelvic inflammatory disease (N. gonorrhoeae, C. trachomatis, BV-associated bacteria), AIDS (HIV-1, HIV-2; HSV, also many opportunistic pathogens), bacterial vaginosis (BV) (BV-associated bacteria), cervicitis (C. trachomatis), cystitis/urethritis (C. trachomatis, N. gonorrhoeae, HSV), enteritis, enterocolitis, epididymitis (C. trachomatis, N. gonorrhoeae), epididymo-orchitis (inflammation of the epididymis and testes) (N. gonorrhoeae), genital and anal warts (Human papillomavirus (genital types), gonococcal dermititis, hepatocellular carcinoma (HBV), Kaposi's sarcoma (HIV), lower genital tract infections: females mucopurulent cervicitis (C. trachomatis, N. gonorrhoeae), lymphoid neoplasia (HIV, HTLV-I), mononucleosis syndrome (Cytomegalovirus, HIV EBV), neoplasias, pharyngitis (N. gonorrhoeae), proctitis (C. trachomatis, N. gonorrhoeae, HSV, T. pallidum), proctocolitis (G. lamblia, Campylobacter spp., Shigella spp., E. histolytica, other enteric pathogens), prostatitis (prostate inflammation) (N. gonorrhoeae), public lice (P. pubis), Reiter's syndrome, salpingitis, scabies (S. scabiei), septicemia, squamous cell cancer of the cervis, anus, vulva, or penis (Human papillomavirus (especially types 16, 18, 31), tropical spastic paraparesis (HTLV-1), ulcerative lesions of the genitalia (HSV-1, T pallidum, H. ducreyi, C. trachomatis (LGV strains), C. granulomatis), urethritis in males (N. gonorrhoeae, C. trachomatis, U urealyticum, USV), urethritis in females (C. trachomatis), vaginitis (C. trachomatis), viral hepatitis (HBV), and vulvovaginitis (C. albicans, T vaginalis). The existence of some forms of STD, for example, trichomonas, in a female subject sometimes result in an imbalance in the endogenous bacteria of the vagina and as a result yeast infections are quite common. Thus, by preventing or treating STDs such as trichomonas, the invention also provides a method for preventing or treating an STD-related yeast infection.

In some embodiments, the invention is not intended to treat certain STD-related conditions such as for example neoplasias or allergies.

In addition, the invention is intended to prevent the transmission of STD infections to the newborns from their infected mothers either in utero or through breast milk. Babies born to mothers infected with chlamydia may suffer from chlamydia eye infections and/or pneumonia. Newborns of mothers infected with N. gonorrhoeae are likely to develop gonococcal ophthalmia. Syphilis can also be transmitted to newborns. Other conditions suffered by newborns born to STD-infected females include conjunctivitis, neurological problems and congenital abnormalities. Thus, by preventing and treating such STD infections in pregnant females, the invention also relates to the prevention of related diseases in offspring born to infected females. STDs in pregnant females can also create complications with pregnancy, including spontaneous abortion, miscarriage, still-born births (syphilis), pre-term delivery (trichomonas), and low birth weight. The invention intends to prevent these latter phenomena by preventing or treating STDs in females who are pregnant or who are at risk of being pregnant.

The nucleic acids are delivered in effective amounts. The term effective amount of a nucleic acid refers to the amount necessary or sufficient to realize a desired biologic effect. For example, an effective amount which alone or in combination with other therapeutics (e.g., an anti-STD agent), and in single or multiple dosages is effective for treatment or prevention of STDs. For instance, when the subject is infected with an STD-causing pathogen an effective amount is that amount which prevents an increase in the number of STD-causing pathogen or which decreases or eliminates all together the infection. This can be assessed using one of the many known diagnostic assays for STD infection (such as those described above). If the subject is not yet infected with an STD-causing pathogen, then an effective amount is that amount which prevents such an infection from arising when the subject is exposed to the organism. Additionally, an effective amount may be that amount which prevents an increase or causes a decrease in a symptom of an STD or which prevents the further development of, or causes a decrease in, an STD-related condition, as described herein. Treatment or prevention of STDs embraces the induction of an immune response either locally (i.e., at a local site at which exposure has is or likely to occur) or systemically. The immune response may include a Th1 response or a Th2 response or a modulation of Th1 and Th2 responses in the subject. Thus, an effective amount is also that amount capable of inducing a local or systemic immune response in the subject.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

The effective amount for any particular application can vary depending on such factors as the type of STD being treated or prevented, the particular nucleic acid being administered (e.g., the number of unmethylated CpG motifs or their location in the nucleic acid), the use of an anti-STD agent or non-drug therapy, the size of the subject, or the severity of the STD or STD-related condition. One of ordinary skill in the art can empirically determine the effective amount of a particular nucleic acid without necessitating undue experimentation.

In embodiments, in which the nucleic acids of the invention are being administered with other therapeutic agents such as, for example, anti STD agents, the effective amount may be that amount of nucleic acid and anti-STD agent which can be administered in combination to achieve the medically beneficial result, as outline above. Thus, it is conceivable that the nucleic acid may be administered in sub-therapeutic amounts, that the anti-STD may be administered in sub-therapeutic amounts or that both may be administered in sub-therapeutic amounts. As an example, in order to treat chlamydia infections, infected subjects are usually administered a seven day schedule of doxycycline, or a single daily dose of azithromycin or five single daily doses of trovafloxacin. When administered with the nucleic acids of the invention, these anti-STD agents may be reduced in dose, or their scheduling may be adjusted so that few doses need be administered.

Similarly, the administration of the nucleic acids of the invention along with anti-STD agents may allow for doses in excess of the maximum tolerated dose of the anti-STD agent to be administered if this is desired. An example of when this latter situation may arise is if the anti-STD agent dose is limited by side effects or by toxicity when administered as a sole agent. Co-administration of the nucleic acid and the anti-STD may allow for a higher dose of the anti-STD to be tolerated by the subject. Administration of the nucleic acid along with an anti-STD agent such as, for example, an antibiotic, may be useful if the subject is allergic to the anti-STD agent. Nucleic acids which are capable of stimulating a Th1 response may be most preferred in this latter embodiment, due to their inherent ability to activate a Th1 response rather than a Th2 response which is detrimental to an allergic reaction.

In embodiments in which the nucleic acids and anti-STD agents of the invention are administered in synergistic combination or when their administration is staggered relative to the other, the STDs to be treated preferably do not include HIV-1, HIV-2, HIV-3, HTLV-I, -II, -III, Hepatitis A, B and C, CMV, HSV-1, HSV-2, HPV, C. trachomatis, Candida albicans, N. gonorrhoeae, and Campylobacter sp.

Subject doses of the compounds described herein typically range from about 0.1 μg to 10 mg per administration, which depending on the application could be given daily, weekly, or monthly and any other amount of time therebetween. More typically mucosal or local doses range from about 10 μg to 5 mg per administration, and most typically from about 100 μg to 1 mg, with 2-4 administrations being spaced hours, days or weeks apart. More typically, immune stimulant doses range from 1 μg to 10 mg per administration, and most typically 10 μg to 1 mg, with daily or weekly administrations. Subject doses of the compounds described herein for parenteral delivery, wherein the compounds are delivered without another therapeutic agent are typically 5 to 10,000 times higher than the effective mucosal dose or for immune stimulant applications, and more typically 10 to 1,000 times higher, and most typically 20 to 100 times higher. More typically parenteral doses for these purposes range from about 10 μg to 5 mg per administration, and most typically from about 100 μg to 1 mg, with 2-4 administrations being spaced hours, days or weeks apart. In some embodiments, however, parenteral doses for these purposes may be used in a range of 5 to 10,000 times higher than the typical doses described above.

In some embodiments, where the nucleic acid has a phosphorothioate backbone, and the disease to be treated is genital warts or other warts caused by Condyloma acuminata, HSV or HPV, the dose of nucleic acid may be, but need not be limited to, less than 6.0 mg/kg/day or less than 3.0 mg/kg/day. Alternatively, in these latter embodiments, the nucleic acids may be administered in doses of 3.0 mg/kg/day or 6.0 mg/kg/day or more and yet administered for less than 14 days, less than 12 days, less than 10 days, less than 8 days, less than 6 days, less than 5 days, and less than three days.

For any compound described herein the therapeutically effective amount can be initially determined from animal models, e.g. the animal models which previously have been described previously. (See J. Infect. Dis. 1999 180(1):203-205; Virology 1996 225(1):213-215; Infect. Immun. 2000 68(1):192-196; J. Infect. Dis. 1999 180(4):1252-8; J. Parasitol. 1998 84(2):321-7.) In vitro assays which are useful in the invention have also been described previously. (See Anotonie Van Leewenhoek 1987; 53 (3):19106; CMAJ 1986 1;135(5):489-93) A therapeutically effective dose can also be determined from human data for CpG nucleic acids which have been tested in humans (human clinical trials have been initiated and the results publicly disseminated) and for compounds which are known to exhibit similar pharmacological activities, such as previously described anti-STD agents, such as those listed herein. Higher doses may be required for parenteral administration, as described above. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the nucleic acid can be administered to a subject by any mode that delivers the nucleic acid to a subject. Administering the pharmaceutical composition of the present invention may be accomplished by any means known to the skilled artisan. Some routes of administration include but are not limited to oral, intranasal, intratracheal, inhalation, ocular, vaginal, rectal, parenteral (e.g. intramuscular, intradermal, intravenous or subcutaneous injection) and direct injection.

For oral administration, the compounds (i.e., nucleic acids and optionally anti-STD agents) can be delivered alone without any pharmaceutical carriers or formulated readily by combining the active compound(s) with pharmaceutically acceptable carriers well known in the art. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions. Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray, from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

When it is desirable to deliver the compounds systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions may also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990, which is incorporated herein by reference.

The nucleic acids and/or anti-STD agents may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The nucleic acids may be delivered in mixtures with anti-STD agent(s). A mixture may consist of several anti-STD agents in addition to the nucleic acid. Alternatively, there may be more than one type of nucleic acid (e.g., a CpG nucleic acid and a T-rich nucleic acid) and one or more anti-STD agents. Additionally, the nucleic acid and the anti-STD agent can be administered with one or more birth control agents or devices.

A variety of administration routes are available. The particular mode selected will depend, of course, upon the particular nucleic acids or anti-STD agents selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some preferred embodiments, the nucleic acids of the invention are administered using a sustained release device, such as those described herein, as well as those known in the art.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides.

Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Sustained release devices, their compositions, their method of manufacture and their release kinetics are known in the art and have been described in a variety of U.S. Patents including those to Epic Therapeutics, Inc., Takeda Chemical Industries. Ltd., ALZA Corp., and Alkermes Control Therapeutics, Inc. Reference can be made to U.S. Pat. Nos. 5,650,173; 5,656,297; 5,679,377; 5,888,533; 5,962,006; 6,110,503; 6,156,331; 6,261,584; 6,265,389; 6,267,981; 6,275,728; 6,268,053; among others.

Sustained release compositions can be applied topically for example as a gel, an ointment, a cream, or a patch (e.g., a transdermal patch or a mucosal patch). As an example, sustained release biodegradable particles can applied to the body surface alone or in the context of an ointment, gel or cream. Topical administration includes administration to a skin surface and a mucosal surface. Mucosal surface delivery can be affected via lipsticks, lip treatments such as lip balms, cold sore ointments; sunscreen ointments; oral gels such as those used for mouth sores (e.g., radiation or chemotherapy induced mouth sores); mouthwashes; toothpaste; inhalants; surface patches; and the like. Alternatively, they can be injected (e.g., subcutaneously, intramuscularly, etc.) or implanted (e.g., Norplant birth control implant). In preferred embodiments, the sustained release devices are biodegradable In other preferred embodiments, the sustained release devices are adhesive to the surface to which they are applied (e.g., skin or mucosa). The art is familiar with such devices.

The nucleic acid may be directly administered to the subject or may be administered in conjunction with a pharmaceutically acceptable carrier or a delivery vehicle. The nucleic acid and optionally other therapeutic agents may be administered alone (e.g. in saline or buffer) or using any delivery vehicles known in the art. One type of delivery vehicle is referred to herein as a nucleic acid delivery complex. A nucleic acid delivery complex shall mean a nucleic acid molecule associated with (e.g. ionically or covalently bound to; or encapsulated within) a targeting means (e.g. a molecule that results in higher affinity binding to target cell (e.g. dendritic cell surfaces and/or increased cellular uptake by target cells). Examples of nucleic acid delivery complexes include nucleic acids associated with: a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to reduce significant uncoupling prior to internalization by the target cell. However, the complex may be cleavable under appropriate conditions within the cell so that the nucleic acid may be released in a functional form.

The nucleic acids may be delivered by non-invasive methods as described above. Non-invasive delivery of compounds is desirable for treatment of children, elderly, animals, and even adults and also to avoid the risk of needle-stick injury. Delivery vehicles for delivering compounds to mucosal surfaces have been described and include but are not limited to: Cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); Liposomes (Childers et al., 1999, Michalek et al., 1989, 1992, de Haan 1995a, 1995b); Live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); Live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); Microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); Polymers (e.g. carboxymethylcellulose, chitosan) (Hamajima et al., 1998, Jabbal-Gill et al., 1998); Polymer rings (Wyatt et al., 1998); Proteosomes (Vancott et al., 1998, Lowell et al., 1988, 1996, 1997); Sodium Fluoride (Hashi et al., 1998); Transgenic plants (Tacket et al., 1998, Mason et al., 1998, Haq et al., 1995); Virosomes (Gluck et al., 1992, Mengiardi et al., 1995, Cryz et al., 1998); Virus-like particles (Jiang et al., 1999, Leibl et al., 1998).

The nucleic acids and/or the other therapeutic agents discussed herein, e.g., anti-STD agents and birth control agents, may also be delivered as a coating on administration devices such as a birth control device (e.g., a condom) or an intravenous bag (e.g., a blood or blood product transfusion bag), intravenous tubing or an intravenous needle. The intravenous bag and/or tubing may be manufactured from sustained release compositions as described above which allow the sustained release of at least the nucleic acids of the invention into the fluid or suspensions contained within the bag and/or tubing.

The invention also embraces kits comprising the nucleic acids of the invention and intended for use in the therapy of subjects in need thereof. The kits contain at a minimum the nucleic acids of the invention, and instructions for use, but preferably also contains other therapeutic agents such as anti-STD agents, birth control agents, and/or birth control devices, or a combination thereof. The nucleic acids may be administered to the mucosal surfaces of the mouth, vagina or anus and rectum by applying the nucleic acids to, for example, the outer surface of a condom prior to or during sexual activity. Thus, the condom may be provided with the nucleic acid on the outer surface, akin to condoms which are prepared with spermicidal compositions on their outer surface. The condom contained within the kit may also have an anti-STD agent on its outer surface and the nucleic acid may be in a separate container. The examples presented herein are intended for illustrative purposes and should not be construed to be limited to condoms only. Rather, any of the birth control devices described herein can be used in a similar fashion.

The kit may also contain the nucleic acid and an anti-STD agent which may or may not be housed in the same container as the nucleic acid. In one embodiment, the kit contains at least one container housing a nucleic acid, an anti-STD agent, and instructions for administering the nucleic acid and the anti-STD agent to a subject having an STD or at risk of developing an STD.

The kit may also contain a birth control agent such as a supply of birth control pills (e.g., a one month supply of birth control pills) or a birth control implant. Preferably, in these latter embodiments, the nucleic acid is already incorporated into the pills or implant. Alternatively, and as an illustrative example, if the pill supply is presented as a circular dial, the nucleic acid may be provided as a separate pill or series of pills contained in a concentric circle either within or outside of the concentric circle housing the birth control pills. This latter embodiment, would be most preferable if the nucleic acid was administered less frequently than the birth control preparation. If the nucleic acid is incorporated into a hormonal implant, the implant may be subdivided such that rather than being commingled, the nucleic acid and the hormone(s) may be released into the subject at different rates. This can be achieved, for example, by placing the nucleic acids and the birth control preparations in different polymer (or other sustained release compositions) with differing rates of diffusion or disintegration.

The following examples are included for purposes of illustration and are not intended to limit the scope of the invention.

### EXAMPLES

The following example illustrates the methodology for demonstrating the ability of a sustained release device, as described above, to deliver nucleic acid topically for the purpose of preventing and/or treating a sexually transmitted disease.

Materials and Methods

Mice: Female C57/B16mice purchased from Charles River, St. Constant, QC are used. In order to synchronize the estrus cycle, mice are injected SC with 2 mg of progesterone per mouse (Depo-Provera; Upjohn, Don Mills, ON), 4 days prior to viral challenge. Placebo sustained release devices (e.g., bioerodible polymer based devices such as mucoadhesive discs) or devices impregnated with 100 μg CpG ODN (sequence #1826: 5′-TCCATGACGTTCCTGACGTT-3′; SEQ ID NO:1) are inserted 24 hrs prior to challenge or at various time-points after challenge (4, 24, 72 hr).

Prophylaxis of HSV-2 infection using BEMA-CpG ODN: Three days following progesterone administration (during diestrus), rolled sustained release discs impregnated with 100 μg CpG ODN (sequence #1826: 5′-TCCATGACGTTCCTGACGTT-3′; SEQ ID NO:1) or rolled control sustained release discs, are inserted such that the bioadhesive side is in contact with the mucosa. Twenty four hours later, mice are swabbed IVAG with a cotton applicator, turned on their backs and infected by IVAG instillation of 10 μl containing 10⁴ PFU HSV-2 (strain 333) during 1 hr while being maintained under halothane anesthesia. Thereafter, mice are washed IVAG daily by pipetting 2×30 μl PBS in and out of vagina 6 to 8 times. Viral titers in vaginal washes are determined by plaque assay on Vero cell monolayers. Genital pathology is monitored daily following HSV-2 challenge and scoring is performed blinded. Pathology is scored on a 5-point scale: 0, no apparent infection; 1, slight redness of external vagina; 2, redness and swelling of external vagina; 3, severe redness and swelling of external vaginal and surrounding tissue; 4, genital ulceration with severe redness, swelling and hair loss of genital and surrounding tissue; 5, severe genital ulceration extending to surrounding tissue. Mice were sacrificed upon reaching stage 5.

Therapy of HSV-2 infection using BEMA-CpG ODN: Three days following progesterone administration mice are infected by IVAG instillation of 10 μI containing 10⁵ PFU HSV-2 during 1 hr as above. At various pre-determined time-points post infection (4, 24, or 72 hr) sustained release discs impregnated with 100 μg CpG ODN (sequence #1826: 5′-TCCATGACGTTCCTGACGTT-3′ SEQ ID NO: 1), or control sustained release discs, are rolled and inserted into the vagina of mice. Thereafter, mice are washed IVAG daily as described above. Viral titers and genital pathology are monitored as above.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments, are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment, of the invention.

All terms used herein are to be given their ordinary meaning, as commonly recognized or as recognized in the art to which they belong, unless otherwise specified.

All references, patents and patent publications that are recited in this application are incorporated in their entirety herein by reference.

What is claimed is:

1. A method for preventing or treating a sexually transmitted disease comprising
administering to a subject in need thereof a poly-G nucleic acid in an amount effective to induce an immune response at a local site in the subject,
wherein the subject is at risk of exposure at the local site to an agent that causes the sexually transmitted disease selected from the group consisting of Neisseria gonorrhoeae, Chlamydia trachomatis, Treponema pallidum, Haemophilus ducreyi, Condyloma acuminata, Calymmatobacterium granulomatis, Ureaplasma urealyticum, Human T lymphotropic virus type I (HTLV-I), Human papilloma virus (multiple types), Hepatitis B virus, Molluscum contagiosum virus, Trichomonas vaginalis, Phthirus pubis, Candida albicans, Mycoplasma hominis, Gardnerella vaginalis and Group B streptococcus, Human T lymphotrophic virus type II (HTLV-II), Hepatitis C and D viruses, Sarcoptes scabiei, Shigella spp., Campylobacter spp., Hepatitis A virus, Giardia lamblia and Entamoeba histolytica.

2. A method for preventing or treating a sexually transmitted disease comprising
administering to a subject in need thereof and not actively exposed to an antigen a poly-G nucleic acid in an amount effective to induce an immune response at a local site in the subject,
wherein the subject is at risk of exposure at the local site to an agent that causes the sexually transmitted disease selected from the group consisting of Herpes simplex virus types 1 and 2 (HSV-1 and HSV-2), Human papilloma virus (multiple types), Hepatitis C and D viruses, and Epstein-Barr virus (EBV).

3. A method for preventing or treating a sexually transmitted disease comprising
administering to a subject in need thereof a non-motif phosphorothioate nucleic acid in an amount effective to induce an immune response at a non-skin local site in the subject,
wherein the subject is at risk of exposure at the local site to an agent that causes the sexually transmitted disease selected from the group consisting of Neisseria gonorrhoeae, Chlamydia trachomatis, Treponema pallidum, Haemophilus ducreyi, Condyloma acuminata, Calymmatobacterium granulomatis, Shigella spp., Ureaplasma urealyticum, Mycoplasma hominis, Gardnerella vaginalis, Campylobacter spp., Group B streptococcus, Human immunodeficiency viruses (HIV-1 and HIV-2), Human T lymphotropic virus type I (HTLV-I), Human T lymphotrophic virus type II (HTLV-II), Herpes simplex virus type I (HSV-1) Herpes simplex virus type 2 (HSV-2), Human papilloma virus (multiple types), Hepatitis A virus, Hepatitis B virus, Hepatitis C and D viruses, Epstein-Barr virus (EBV), Cytomegalovirus and Molluscum contagiosum virus, Trichomonas vaginalis, Sarcoptes scabiei, Giardia lamblia, Phthirus pubis, Entamoeba histolytica and Candida albicans.

4. A method for preventing or treating a sexually transmitted disease comprising
administering to a subject in need thereof a non-motif phosphorothioate nucleic acid in an amount effective to induce an immune response at a local site in the subject,
wherein the subject is at risk of exposure at the local site to an agent that causes the sexually transmitted disease selected from the group consisting of Neisseria gonorrhoeae, Chlamydia trachomatis, Treponema pallidum, Haemophilus ducreyi, Condyloma acuminata, Calymmatobacterium granulomatis, Shigella spp., Ureaplasma urealyticum, Mycoplasma hominis, Gardnerella vaginalis, Campylobacter spp., Group B streptococcus, Human immunodeficiency viruses (HIV-1 and HIV-2), Human T lymphotropic virus type I (HTLV-I), Human T lymphotrophic virus type II (HTLV-II), Hepatitis A virus, Hepatitis B virus, Hepatitis C and D viruses, Epstein-Barr virus (EBV), Cytomegalovirus and Molluscum contagiosum virus, Trichomonas vaginalis, Sarcoptes scabiei, Giardia lamblia, Phthirus pubis, Entamoeba histolytica and Candida albicans.

5. A method for preventing or treating a sexually transmitted disease, comprising
administering to a subject in need thereof a nucleic acid in an amount effective to induce an immune response at a local site in the subject,
wherein the subject is at risk of exposure at the local site to an agent that causes the sexually transmitted disease selected from the group consisting of Haemophilus ducreyi, Calymmatobacterium granulomatis, Ureaplasma urealyticum, Gardnerella vaginalis, Shigella spp., Molluscum contagiosum virus, Epstein-Barr virus, Trichomonas vaginalis, Phthirus pubis, Giardia lamblia, Entamoeba histolytica, and Sarcoptes scabiei.

6. The method of claim 1, 2, 3, 4 or 5, wherein the subject is not actively exposed to an antigen.

7. The method of claim 1, 2, 3, 4 or 5, wherein the nucleic acid is administered to the subject prior to engaging in a high risk activity.

8. The method of claim 1, 2, 3, 4 or 5, wherein the nucleic acid is administered to the subject during a high risk activity.

9. The method of claim 1, 2, 3, 4 or 5, wherein the nucleic acid is administered to the subject following a high risk activity.

10. The method of claim 6, 7 or 8, wherein the high risk activity is selected from the group consisting of sexual intercourse, blood transfusion, intravenous needle use, childbirth, and medical procedures.

11. The method of claim 6, 7 or 8, wherein the high risk activity is a blood transfusion and the nucleic acid is coated on an inside surface of a transfusion bag or an intravenous tube or an intravenous needle.

12. The method of claim 6, 7 or 8, wherein the high risk activity is sexual intercourse and the nucleic acid is coated on a birth control device.

13. The method of claim 5, wherein the nucleic acid is an immunostimulatory CpG nucleic acid having an unmethylated CpG motif.

14. The method of claim 5, wherein the nucleic acid is an immunostimulatory T-rich nucleic acid.

15. The method of claim 5, wherein the nucleic acid is an immunostimulatory poly G nucleic acid.

16. The method of claim 5, wherein the nucleic acid is an immunostimulatory methylated CpG nucleic acid having a methylated CpG motif.

17. The method of claim 1, 2, 3, 4 or 5, further comprising administering an anti-STD agent.

18. The method of claim 17, wherein the anti-STD agent is an anti-bacterial agent.

19. The method of claim 17, wherein the anti-STD agent is an anti-viral agent.

20. The method of claim 1, 2, 3, 4 or 5, wherein the nucleic acid is not an anti-sense nucleic acid.

21. The method of claim 1, 2 or 5, wherein the nucleic acid has a modified backbone.

22. The method of claim 21, wherein the modified backbone is a phosphate backbone modification.

23. The method of claim 1, 2, 3, 4 or 5, wherein the nucleic acid is administered in a birth control device.

24. The method of claim 23, wherein the birth control device is a selected from the group consisting of a condom, an intra-uterine device, an intra-vaginal device, a cervical cap, a diaphragm, and a sponge.

25. The method of claim 1, 2, 3, 4 or 5, wherein the nucleic acid is administered with a birth control agent.

26. The method of claim 25, wherein the birth control agent is selected from the group consisting of a birth control pill, a birth control implant, a morning after pill, and a spermicide.

27. The method of claim 1, 2, 3, 4 or 5, wherein the local site is selected from the group consisting of mouth, vagina, anus, penis, eye and blood vessel.

28. The method of claim 1, 2, 3, 4 or 5, wherein the local site is not a mucosal surface.

29. The method of claim 1, 2, 3, 4 or 5, wherein the nucleic acid is administered in a sustained release device.

30. The method of claim 29, wherein the sustained release device is selected from the group consisting of a polymer based sustained release device, a non-polymer sustained release device, an intravenous bag, a suppository, a mucosal patch, and an implant.

31. The method of claim 29, wherein the sustained release device is a wall of the intravenous bag, is in a wall of the intravenous bag, or is in an intravenous bag.

32. A non-vaccine composition, comprising:
a CpG nucleic acid formulated in a sustained release device in an effective amount, wherein the nucleic acid does not encode a peptide or polypeptide.

33. A composition, comprising:
a nucleic acid selected from the group consisting of a poly-G nucleic acid and a non-motif phosphorothioate nucleic acid, formulated in a sustained release device in an effective amount.

34. The non-vaccine composition of claim 32 or 33, wherein the sustained release device is selected from the group consisting of a polymer-based sustained release device, a non-polymer based sustained release device, a microparticle, a microcapsule, a hydrogel, a rozinger, a pessary.

35. A composition comprising
a nucleic acid in an a pharmaceutically acceptable carrier and in an effective amount, and
a birth control agent.

36. A composition comprising
a nucleic acid in an a pharmaceutically acceptable carrier and in an effective, and
a birth control device.

37. A composition comprising
a nucleic acid, and
an intravenous bag, wherein the nucleic acid is situated within the intravenous bag.

38. A composition comprising
a nucleic acid, and
a diaper, wherein the nucleic acid is contained within or on the surface of the diaper.

39. A kit comprising
the composition of claim 32, 33, 35, 36, 37, or 38, and
instructions for administering the composition to a subject having or at risk of developing an STD.


SEQUENCE LISTING (1 sequence)

SEQ ID NO: 1 - DNA, 20 bp, Artificial Sequence
  Synthetic Sequence
tccatgacgttcctgacgtt